(19) United States
(12) Patent Application Publication
(10) Pub. No.: US 2024/0408308 A1
(43) Pub. Date: Dec. 12, 2024

(54) CHARACTERIZATION APPARATUS FOR DRUG DELIVERY DEVICES OR SUBCOMPONENTS THEREOF

(71) Applicant: AMGEN INC., Thousand Oaks, CA (US)

(72) Inventors: Seyed Reza Mirhassani Moghaddam, Thousand Oaks, CA (US); Julian Jazayeri, Woodland Hills, CA (US); Sandra Sanchez, Thousand Oaks, CA (US); Edgardo Halili, Santa Clara, CA (US); Cindy Saldana, Thousand Oaks, CA (US); Sergio Giovanni Valenzuela, Winnetka, CA (US)

(21) Appl. No.: 18/697,770

(22) PCT Filed: Sep. 30, 2022
(86) PCT No.: PCT/US2022/045303

§ 371 Date: Apr. 2, 2024

Related U.S. Application Data
(60) Provisional application No. 63/254,294, filed on Oct. 11, 2021.

(51) Int. Cl. A61M 5/20; G01L 5/00; G16H 20/17

(52) U.S. Cl. CPC A61M 5/20; G01L 5/0038; G16H 20/17; A61M 2005/2093; A61M 2205/332

(57) ABSTRACT

Apparatuses, systems and methods are provided that characterize a rear sub-assembly for an autoinjector (AI). Apparatuses, systems and methods are also provided that generate injection time (IT) models for autoinjectors (AIs). Autoinjectors (AIs) (e.g., mechanical AIs, spring-loaded AIs, etc.), components for use within AIs (e.g., prefilled syringes (PFS), rear sub-assemblies (RSAs), etc.), and related methods may utilize mathematical modeling along with design for six sigma (DFSS) and design for reliability and manufacturability (DRM) to establish upstream controls that ensure injection time (IT) robustness of mechanical autoinjectors (AIs). For example, break-loose and extrusion (BLE) alert limit, rear sub-assembly (RSA) area under the curve (AUC), and RSA minimum force.

### CROSS-REFERENCE TO RELATED APPLICATION

Priority is claimed to U.S. Provisional Patent Application No. 63/254,294, filed Oct. 11, 2021, the entire contents of which are hereby incorporated by reference herein.

### FIELD OF DISCLOSURE

The present disclosure generally relates to automatic drug product injectors. More specifically, the present disclosure relates to generating injection time (IT) models for autoinjectors (AI) based on prefilled syringe (PFS) data and rear sub-assembly (RSA) data.

### BACKGROUND

Numerous drug products are manufactured and packaged in, for example, an autoinjector (AI). AIs may include a prefilled syringe (PFS) and a rear sub-assembly (RSA) for providing force to automatically extrude a drug product (DP) from the PFS when the AI is activated by a user.

Injection time (IT) is often specified by, for example, a DP manufacture. Achieving a consistent IT is often problematic in known AIs. Selection of a RSA for a particular PFS is difficult at best. Thus, known AIs often fail to robustly achieve a specified injection time.

Apparatuses, systems and methods are also needed that characterize a rear sub-assembly for an autoinjector (AI). Apparatuses, systems and methods are needed that generate injection time (IT) models for autoinjectors (AIs).

### SUMMARY

A rear sub-assembly (RSA) output force characterization method may include providing a syringe-free (SyFR) rear sub-assembly (RSA) operating apparatus. The method may also include generating RSA output force profile data using the syringe-free (SyFR) rear sub-assembly (RSA) operating apparatus. The RSA output force profile data may be representative of a rear sub-assembly (RSA) output force profile. The method may further include generating a RSA output energy profile based on the RSA output force profile data.

In another embodiment, an apparatus for characterizing output force of a drug delivery device or a rear subassembly of a drug delivery device may include a driver holder configured to secure the drug delivery device or the rear subassembly to the apparatus. The drug delivery device or the rear subassembly may include a drive member having a compressed position and an extended position. The apparatus may also include a force sensor configured to measure output force data associated with the drive member as the drive member moves from the compressed position to the extended position. The apparatus may further include an extrusion speed input configured to receive extrusion speed data. The extrusion speed data may be representative of a speed of the drive member. The apparatus may yet further include a controller configured to receive the output force data and the extrusion speed input data, and to generate an output force profile based on the output force data. The controller may be further configured to generate an output energy profile based on the output force profile data.

In a further embodiment, a non-transitory computer-readable medium storing computer-readable instructions that, when executed by a processor, causes the processor to generate an injection time (IT) model for an autoinjector. Execution of the instructions may cause the processor to receive prefilled syringe (PFS) break loose and extrusion resistance (BLER) data. The BLER data may be representative of an amount of force required to achieve respective extrusion speeds. Further execution of the instructions may cause the processor to receive rear sub-assembly (RSA) output force profile data. Further execution of the instructions may cause the processor to generate an injection time (IT) model for the autoinjector based on the BLER data and the RSA output force profile data.

### BRIEF DESCRIPTION OF THE DRAWINGS

It is believed that the disclosure will be more fully understood from the following description taken in conjunction with the accompanying drawings. Some of the drawings may have been simplified by the omission of selected elements for the purpose of more clearly showing other elements. Such omissions of elements in some drawings are not necessarily indicated of the presence or absence of particular elements in any of the exemplary embodiments, except as may be explicitly delineated in the corresponding written description. Also, none of the drawings are necessarily to scale.

FIG. 1 depicts a block diagram of an example injection time (IT) model for an autoinjector (AI);

FIGS. 2A-C depict various views of an example autoinjector (AI);

FIG. 3 depicts an example system for generating an IT model for an autoinjector (AI);

FIG. 4 depicts an example rear sub-assembly (RSA) test fixture;

FIGS. 5A-E depict an example system for generating an IT model for an autoinjector (AI);

FIG. 6 depicts an example break loose and extrusion resistance (BLER) profile of a prefilled syringe (PFS);

FIG. 7 depicts an example rear sub-assembly (RSA) output force profile;

FIG. 8 depicts an example force balance analysis used for an IT model;

FIG. 9 depicts an example hypothetical maximum prefilled syringe (PFS) break loose and extrusion resistance (BLER) profile; and

FIG. 10 depicts an example minimum energy area under the curve (AUC) for drug product extrusion

Skilled artisans will appreciate that elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercial feasible embodiment are often not depicted in order to facilitate a less obstructed view of these various embodiments. It will further be appreciated that certain actions and/or steps may be described or depicted in a particular order of occurrence while those skilled in the art will understand that such specificity with respect to sequence is not actually required. It will further be appreciated that certain actions and/or steps may be described or depicted in a particular order of occurrence while those skilled in the art will understand that such specificity with respect to sequence is not actually required. It will also be understood that the terms and expressions used herein have the ordinary technical meaning as is accorded to such terms and expressions by persons skilled in the technical field as set forth above except where different specific meanings have otherwise been set forth herein.

### DETAILED DESCRIPTION

Apparatuses, systems and methods are provided that characterize a rear sub-assembly for an autoinjector (AI). Apparatuses, systems and methods are also provided that generate injection time (IT) models for autoinjectors (AIs).

Autoinjectors (AIs) (e.g., mechanical AIs, spring-loaded AIs, etc.), components for use within AIs (e.g., prefilled syringes (PFS), rear sub-assemblies (RSAs), etc.), and related methods may utilize mathematical modeling along with design for six sigma (DFSS) and design for reliability and manufacturability (DRM) to establish upstream controls that ensure injection time (IT) robustness of mechanical autoinjectors (AIs). For example, break-loose and extrusion (BLE) alert limit, rear sub-assembly (RSA) area under the curve (AUC), RSA minimum force, etc. may be determined, and used to select a RSA for any given PFS.

Upstream controls (e.g., break loose and extrusion (BLE) alert limits, an area under the curve (AUC), a stall force, etc.) may be established to ensure robustness of injection time (IT) performance for an autoinjector (AI) (e.g., a mechanical AI, a spring-loaded mechanical AI, etc.). These controls may ensure quality of AIs, and may improve customer experience by bridging the gap between component specifications (e.g., PFS specifications, RSA specifications, etc.) and user specifications (e.g., injection time (IT), etc.). A sustainable and repeatable AI design framework is provided that is transferrable to future drug products (DPs) and autoinjectors (AIs).

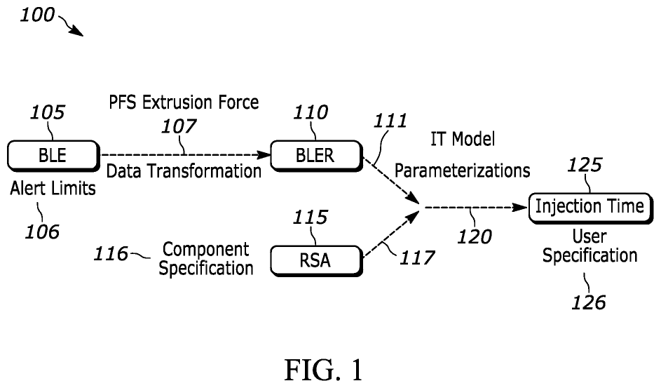

Turning to FIG. 1, a block diagram of an injection time (IT) model generation system 100 may include break loose and extrusion (BLE) 105 alert limits 106, prefilled syringe (PFS) extrusion forces 107 data transformation, break loose and extrusion resistance (BLER) 110 with IT model parameterizations 111, rear sub-assembly 115 with component specification 116 and RSA characterization profile data 117 combined as input 120 to injection time (IT) 125 having a user specification 126. The injection time (IT) model generation system 100 may predict IT performance of an AI using a mathematical model as, for example, described in detail herein. Model generation system 100 may predict IT using empirical data of a prefilled syringe (PFS) and a rear sub-assembly (RSA). Therefore, the model generation system 100 may readily connect component (PFS and RSA) specifications to user specification (IT). PFS resistance may be quantified using BLE and Break-loose and Extrusion Resistance (BLER) force. RSA mechanical performance may be characterized using RSA output force measurement. Details in regard to a mathematical model for IT are included herein.

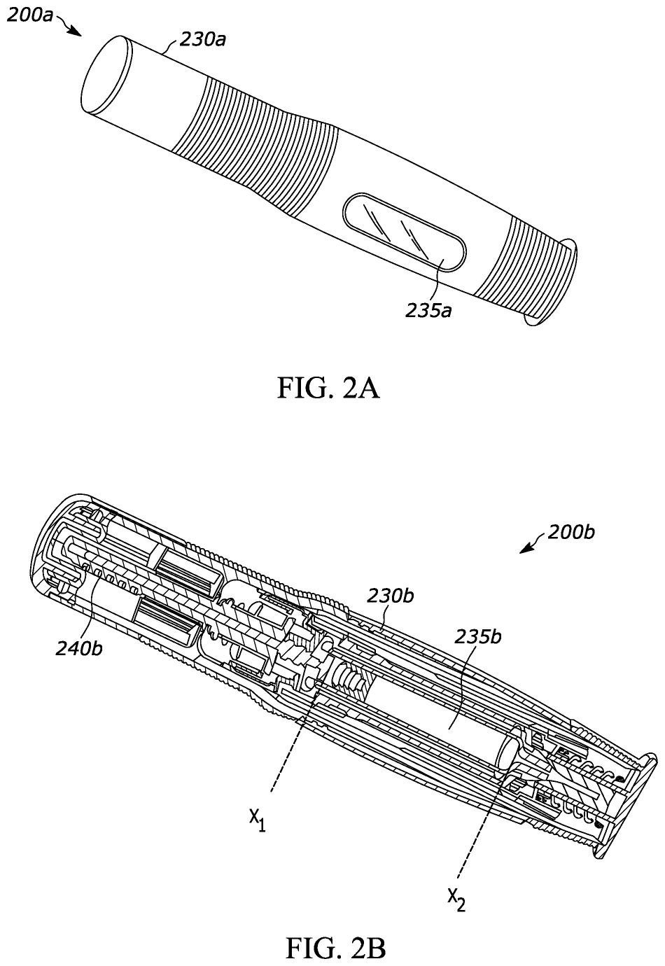

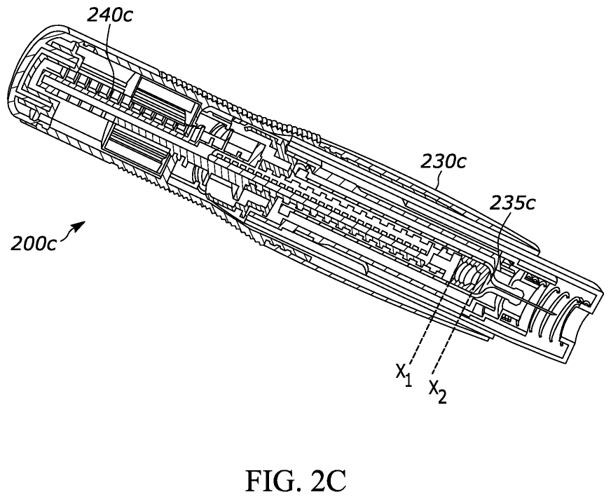

With reference to FIGS. 2A-C, an injection time (IT) model generation system 200a-c may include an autoinjector (AI) 230a-c (e.g., a single-use spring-loaded AI, etc.) having a prefilled syringe (PFS) 235a-c (and a rear sub-assembly (RSA) 240a-c (e.g., a mechanical RSA, a spring-loaded RSA, etc.).

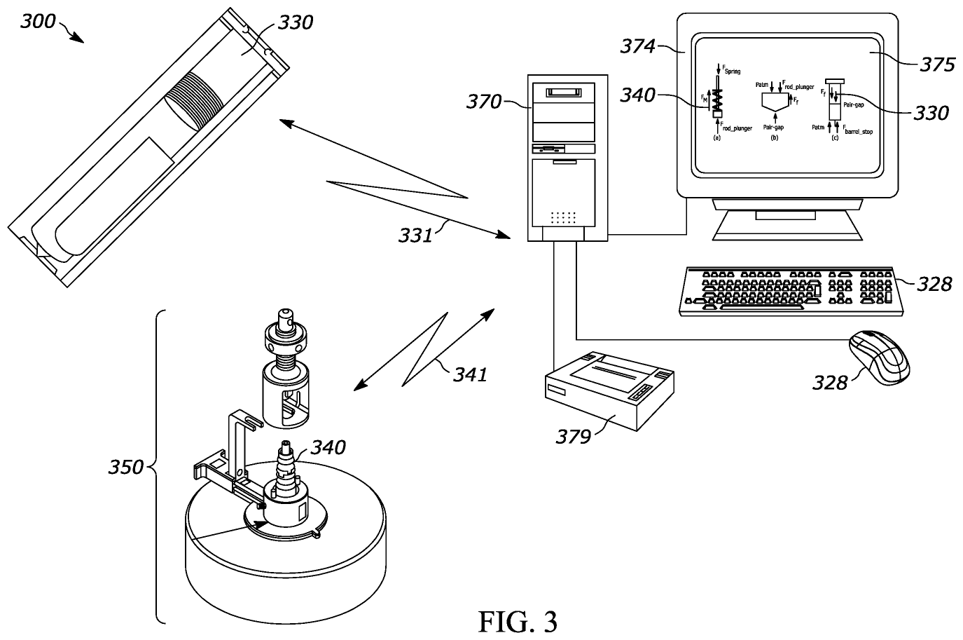

Turning to FIG. 3, an autoinjector (AI) injection time (IT) model generation system 300 may include receiving break loose and extrusion force data 331 for a prefilled syringe (PFS) 330 and rear sub-assembly (RSA) characterization data 341 from a RSA 340 and a RSA fixture 350. The autoinjector injection time (IT) model generation system 300 may also include a remote device 370 having using input devices 328, a display device 374, and a printer 379. The display device 374 may include a user interface 375 configured to, for example, generate an injection time (IT) model for an autoinjector (AI).

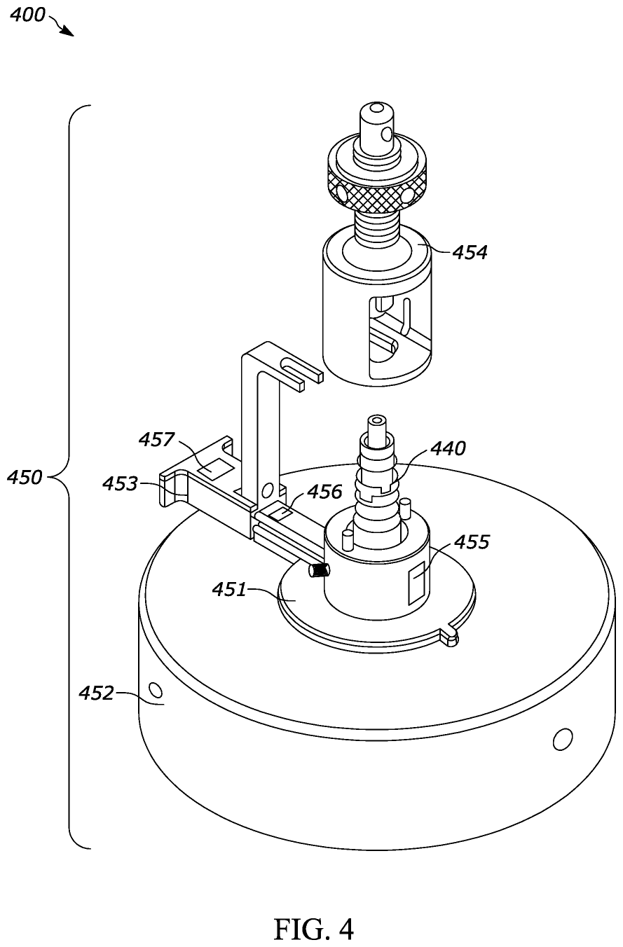

With reference to FIG. 4, an autoinjector (AI) injection time (IT) model generation system 400 may include a rear subassembly (RSA) fixture 450 (e.g., a syringe-free (SyFR) RSA testing fixture, etc.) and a rear sub-assembly (RSA) 440. The RSA fixture 450 may include a base 451, a discoid 452, a syringe driver holder 453, and a RSA load applicator 454. The RSA fixture may generate rear sub-assembly (RSA) characterization data from the load applicator 454 and the RSA 440.

The rear subassembly fixture 450 may be configured to characterize output force of a drug delivery device or a rear subassembly of a drug delivery device. The fixture 450 may include a driver holder 453 configured to secure the drug delivery device or the rear subassembly to the fixture 450. The drug delivery device or the rear subassembly may include a drive member having a compressed position and an extended position. The apparatus may also include a force sensor 456 configured to measure output force data associated with the drive member as the drive member moves from the compressed position to the extended position. The fixture 450 may further include an extrusion speed sensor 457 configured to provide an extrusion speed of the drive member. The fixture 450 may yet further include a controller 455 configured to receive the output force data from the sensor 456 and to generate an output force profile based on the output force data. The controller 455 may be further configured to generate an output energy profile based on the output force profile data and extrusion speed data.

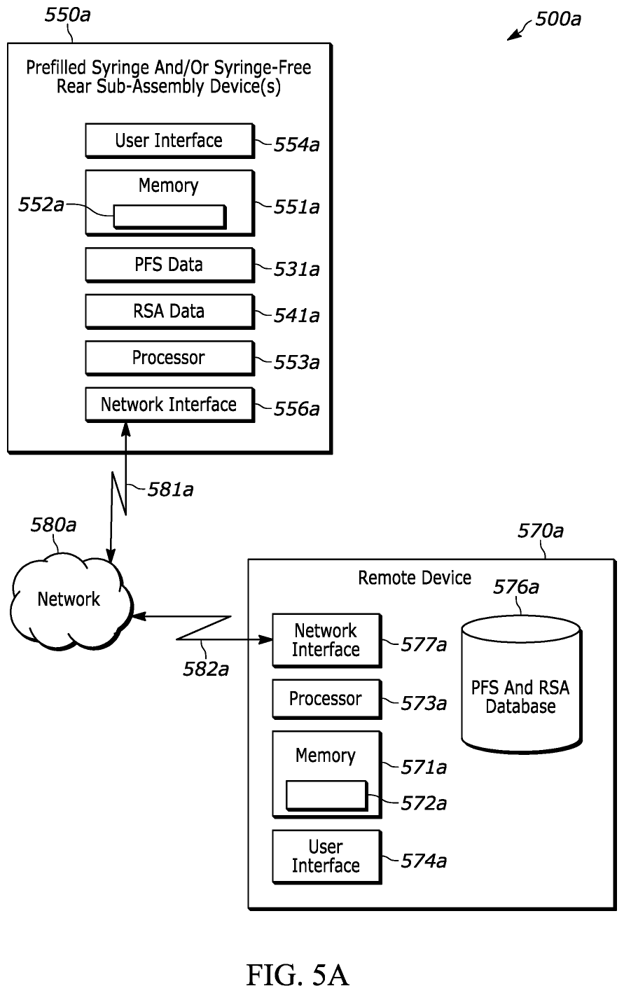

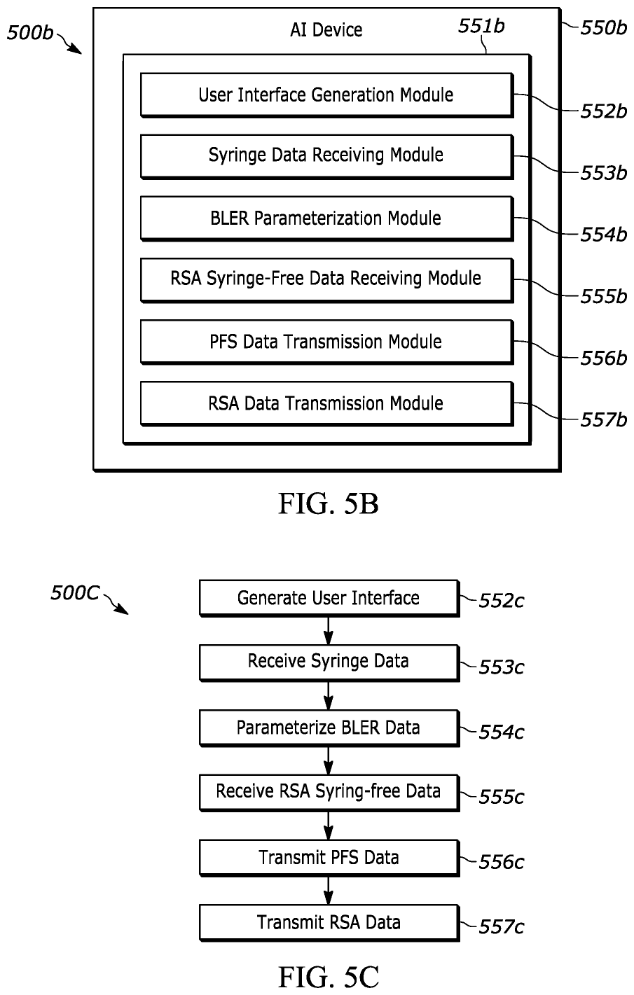

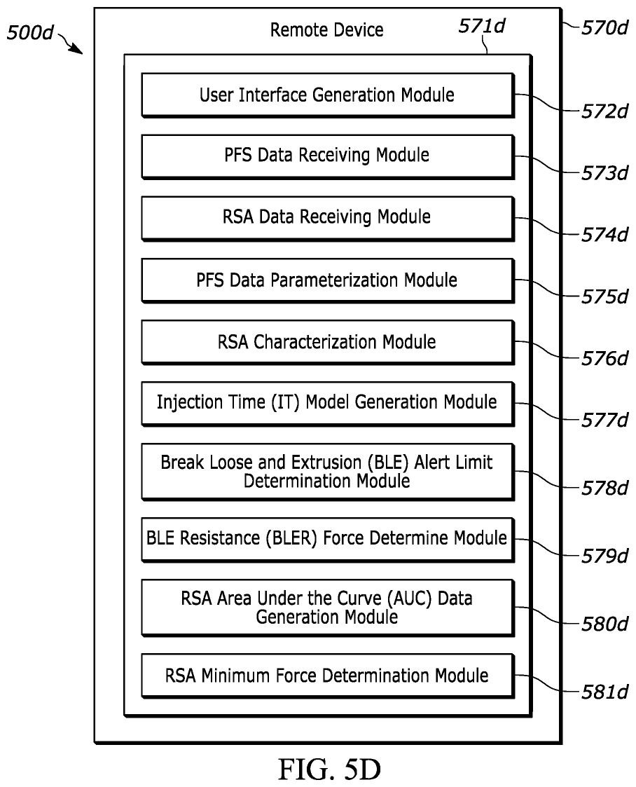

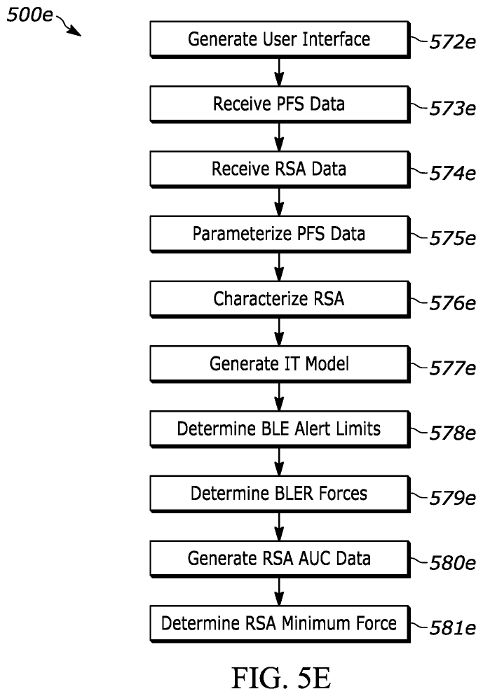

Turning to FIGS. 5A-E, an autoinjector injection time (IT) model generation system 500a-e may include a prefilled syringe (PFS) and/or syringe-free rear sub-assembly device 550a-c in communication with a remote device (e.g., a server) 570a,d,e via a network 580a. The prefilled syringe (PFS) and/or syringe-free rear sub-assembly device 550a-c may be similar to, for example, the prefilled syringe (PFS) and/or syringe-free rear sub-assembly device 350 of FIG. 3. The remote device 570a,d,e may be similar to, for example, the remote device 120 of FIG. 1.

The autoinjector injection time (IT) model generation system 500a-e may implement communications between the prefilled syringe (PFS) and/or syringe-free rear sub-assembly device 550a-c and the remote device 570a,d,e (e.g., a remote server, cloud-based resources, etc.) to provide, for example, RSA data and/or PFS data to a PFS and RSA database 576a.

For example, the autoinjector injection time (IT) model generation system 500a-e may acquire prefilled syringe data (e.g., prefilled syringe physical dimension data, prefilled syringe optical transmission data, prefilled syringe manufacture data, etc.) from, for example, a user of a prefilled syringe (PFS) and/or syringe-free rear sub-assembly device 550a-c. Alternatively, or additionally, while not shown in FIGS. 5A-E, syringe data and/or BLER data may be automatically obtained from a third party data source (e.g., a syringe manufacture, a medication manufacturer, etc.). As described in detail herein, the autoinjector injection time (IT) model generation system 500a-e may automatically generate an injection time (IT) model for an autoinjector (AI) based on, for example, BLER data and RSA characterization data.

For clarity, only one prefilled syringe (PFS) and/or syringe-free rear sub-assembly device 550a-c is depicted in FIG. 5A. While FIG. 5A depicts only one prefilled syringe (PFS) and/or syringe-free rear sub-assembly device 550a-c, it should be understood that any number of prefilled syringe (PFS) and/or syringe-free rear sub-assembly device 550a-c may be supported. A prefilled syringe (PFS) and/or syringe-free rear sub-assembly device 550a-c may include a memory 551a and a processor 553a for storing and executing, respectively, a module 552a. The module 552a stored in the memory 551a as a set of computer-readable instructions, may be related to an application for automatically generating an IT model for an AI.

As described in detail herein, the module 552a may facilitate interaction between an associated prefilled syringe (PFS) and/or syringe-free rear sub-assembly device 550a-c and a remote device 570a,d,e. For example, the processor 553a, further executing the module 552a, may facilitate communications between a remote device 570a,d,e and a prefilled syringe (PFS) and/or syringe-free rear sub-assembly device 550a-c via a network interface 556a, a communication link 581a, a network 580a, a remote device communication link 582a, and a remote device network interface 577a.

A prefilled syringe (PFS) and/or syringe-free rear sub-assembly device 550a-c may include a user interface 554a which may be any type of electronic display device, such as touch screen display, a liquid crystal display (LCD), a light emitting diode (LED) display, a plasma display, a cathode ray tube (CRT) display, or any other type of known or suitable electronic display along with a user input device. A user interface 554a may exhibit a user interface (e.g., any user interface 375, etc.) which depicts a user interface for configuring a prefilled syringe (PFS) and/or syringe-free rear sub-assembly device 550a-c to communicate with a remote device 570a,d,e.

The network interface 580a may be configured to facilitate communications between a prefilled syringe (PFS) and/or syringe-free rear sub-assembly device 550a-c and a remote device 570a,d,e via any wireless communication network 580a, including for example a wireless LAN, MAN or WAN, WiFi, the Internet, or any combination thereof. Moreover, a prefilled syringe (PFS) and/or syringe-free rear sub-assembly device 550a-c may be communicatively connected to a remote device 570a,d,e via any suitable communication system, such as via any publicly available or privately owned communication network, including those that use wireless communication structures, such as wireless communication networks, including for example, wireless LANs and WANs, satellite and cellular telephone communication systems, etc. A prefilled syringe (PFS) and/or syringe-free rear sub-assembly device 550a-c may cause, for example, prefilled syringe data and/or RSA data to be transmitted to, and stored in, for example, a remote device 570a,d,e, memory 571a, and/or a remote PFS and RSA database 576a.

The prefilled syringe (PFS) and/or syringe-free rear sub-assembly device 550a-c may include a PFS data source 531a and a RSA data source 541a. As described in detail herein, the prefilled syringe (PFS) and/or syringe-free rear sub-assembly device 550a-c may be configured to, for example, cause the processor 543a to acquire PFS data 331 and/or RSA data 341.

A remote device 450b,e may include a user interface 574a, a memory 571a, and a processor 573a for storing and executing, respectively, a module 572a. The module 572a, stored in the memory 571a as a set of computer-readable instructions, may facilitate applications related to automatically generating an IT model for an AI. The module 572a may also facilitate communications between the remote device 570a,d,e and a prefilled syringe (PFS) and/or syringe-free rear sub-assembly device 550a-c via a network interface 577a, and the network 580a, and other functions and instructions.

A remote device 570a,d,e may be communicatively coupled to a prefilled syringe (PFS) and/or syringe-free rear sub-assembly device 550a-c. While the prefilled syringe (PFS) and/or syringe-free rear sub-assembly device 550a is shown in FIG. 5A as being communicatively coupled to the remote device 570a, it should be understood that the PFS and RSA database 576a may be located within separate remote servers (or any other suitable computing devices) communicatively coupled to the remote device 570a,d,e. Optionally, portions of PFS and RSA database 576a may be associated with memory modules that are separate from one another, such as a memory 551a of a prefilled syringe (PFS) and/or syringe-free rear sub-assembly device 550a-c.

A prefilled syringe (PFS) and/or syringe-free rear sub-assembly device 550a-c may include a user interface generation module 552b, a prefilled syringe data receiving module 553b, a BLER parameterization module 554b, a RSA syringe-free data receiving module 555b, a PFS data transmission module 556b, and a RSA data transmission module 557b, for example, stored on a memory 551b as a set of computer-readable instructions. In any event, the modules 552b-557b may be similar to, for example, the module 552a of FIG. 5A.

A method of operating a prefilled syringe (PFS) and/or syringe-free rear sub-assembly device 550a-c may be implemented by a first processor (e.g., processor 553a) executing, for example, at least a portion of modules 552b-557b. In particular, processor 553a may execute the user interface generation module 552b to cause the processor 553a to, for example, generate a user interface 375 (block 552e). The user interface may allow a user to enter, for example, prefilled syringe data and/or RSA data.

Processor 553a may execute the syringe data receiving module 553b to cause the processor 553a to, for example, receive prefilled syringe data from a prefilled syringe manufacture, a medicament manufacture, etc. (block 553c). Processor 553a may execute the BLER parameterization module 554b to cause the processor 553a to, for example, parameterize the syringe data (block 554c). Processor 553a may execute the RSA syringe-free data receiving module 555b to cause the processor 553a to, for example, receive RSA syringe-free data (block 555c). Processor 553a may execute the PFS data transmission module 556b to cause the processor 553a to, for example, transmit PFS data (block 556c). Processor 553a may execute the RSA data transmission module 557b to cause the processor 553a to, for example, transmit RSA data (block 556c).

A remote device 570a,d,e may include a user interface generation module 572d, a syringe data receiving module 573d, a RSA data receiving module 574d, a PFS data parameterization module 575d, a RSA characterization module 576d, an injection time (IT) model generation module 577d, a break loose and extrusion (BLE) Alert limit determination module 578d, a BLE resistance (BLER) force determination module 579d, a RSA area under the curve (AUC) data generation module 580d, and a RSA minimum force determination module 581d, for example, stored on a memory 571d as a set of computer-readable instructions. In any event, the modules 572d-581d may be similar to, for example, the module 572a of FIG. 5A.

A method of operating a remote device 500e may be implemented by a processor (e.g., processor 573a) executing, for example, at least a portion of modules 572d-581d. In particular, processor 573a may execute the user interface generation module 572d to cause the processor 573a to, for example, generate a user interface 375, etc. (block 572e).

Processor 573a may execute the syringe data receiving module 573d to cause the processor 573a to, for example, receive prefilled syringe data from a user via a user interface and/or from a third-party prefilled syringe database (block 573e). Processor 573a may execute the RSA data receiving module 574d to cause the processor 573a to, for example, receive RSA data (block 574e).

Processor 573a may execute the PFS data parameterization module 575d to cause the processor 573a to, for example, parameterized PFS data (block 575e). Processor 573a may execute the RSA characterization module 576d to cause the processor 573a to, for example, characterize a RSA (block 576e). Processor 573a may execute the injection time (IT) model generation module 577d to cause the processor 573a to, for example, generate an IT model for an AI (block 577e). Processor 573a may execute the break loose and extrusion (BLE) Alert limit determination module 578d to cause the processor 573a to, for example, to determine break loose and extrusion (BLE) Alert limits (block 578e).

Processor 573a may execute the BLE resistance (BLER) force determination module 579d, to cause the processor 573a to, for example, determine BLER force (block 579e). Processor 573a may execute the RSA area under the curve (AUC) data generation module 580d to cause the processor 573a to, for example, generate RSA AUC data (block 580e). Processor 573a may execute the RSA minimum force determination module 581d to cause the processor 573a to, for example, determine RSA minimum force (block 581e).

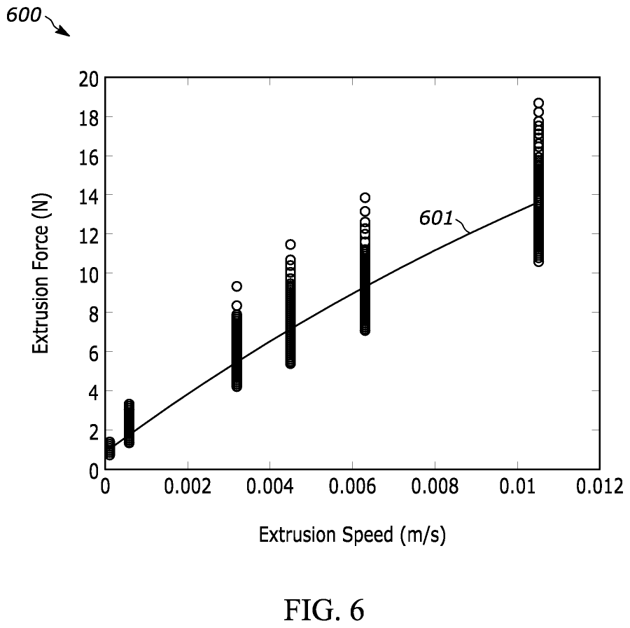

With reference to FIG. 6, an injection time (IT) model generation system 600 may generate an IT model based on data used to generate graphical representation 601 of forces that were measured using a BLER method for a Repatha PFS. This test method may characterize injection behavior of a single PFS by performing the test across various speeds within a single syringe. Test speeds used therein may be representative of speed profiles that occur during, for example, injection of drug product (DP) via a spring loaded mechanical autoinjector (AI). Having the resistance of a PFS fully characterized at various speeds allows for defining BLE alert limits via, for example, a mathematical model of injection time (IT). Moreover, having this (BLER) characterization performed on a PFS, may provide greater insight into how PFS extrusion force is related to AI output force.

Utilizing the prefilled syringe data that the BLER test method collects, BLER profiles (e.g., BLER profiles 601 of FIG. 6, etc.) may be parameterized in order to be used within a mathematical model of injection time (IT). Prefilled syringe (PFS) BLER profiles may be, for example, parameterized as a quadratic function of extrusion speed (e.g., data illustrated in graph 601 of FIG. 6, etc.). The BLER force profile (F_{pfs}) may be parameterized via:

$${F}_{pfs} = {{C}_{1} ( \frac{dx}{dt} )}^{2} + {C}_{2} \frac{dx}{dt} + {F}_{f} \qquad Eq . 1$$

In Eq. 1.,

$$\frac{dx}{dt}$$

is representative of the extrusion speed; C₁, C₂, and Fr represent the 2^{nd} order and 1^{st} order damping coefficients and low speed glide force of the PFS, respectively. C₂ is demonstrative of the non-Newtonian behavior of the DP in the syringe. If C₂ is greater than zero, a shear-thickening behavior for the DP is inferred. On the other hand, a shear-thinning behavior of the DP is inferred if C₂ is smaller than zero. If C₂ is close to zero, then a linear curve is expected and therefore a Newtonian behavior for the DP is inferred. Fr represents the low speed glide force of the PFS and is inferred as the amount of force needed to keep the plunger-stopper traveling during the injection and avoid stall of an associated AI.

Eq. 1 may be used within a mathematical model of IT for predicting IT. A BLER method may provide quantitative measurements of an amount of force required for extrusion of a drug product (DP) at various extrusion speeds that occur throughout a full dose injection.

A rear sub-assembly (RSA) output force characterization method (e.g., a Syringe-free (SyFR) release testing method, etc.) may collect quantitative information on output force of the RSA in the form of, for example, area under the curve (AUC), minimum force, pre-activation force and activation force with only the RSA of an AI present. These measurements may be used for predicting injection time (IT) of an associated autoinjector (AI). An SyFR method may not constrain movement of an actuator sleeve of an associated rear sub-assembly (RSA), and may allow for more accurate measurements by fully taking into consideration frictional forces that may occur during movement of a plunger rod of the RSA. The SyFR method may be applied to all mechanical AIs.

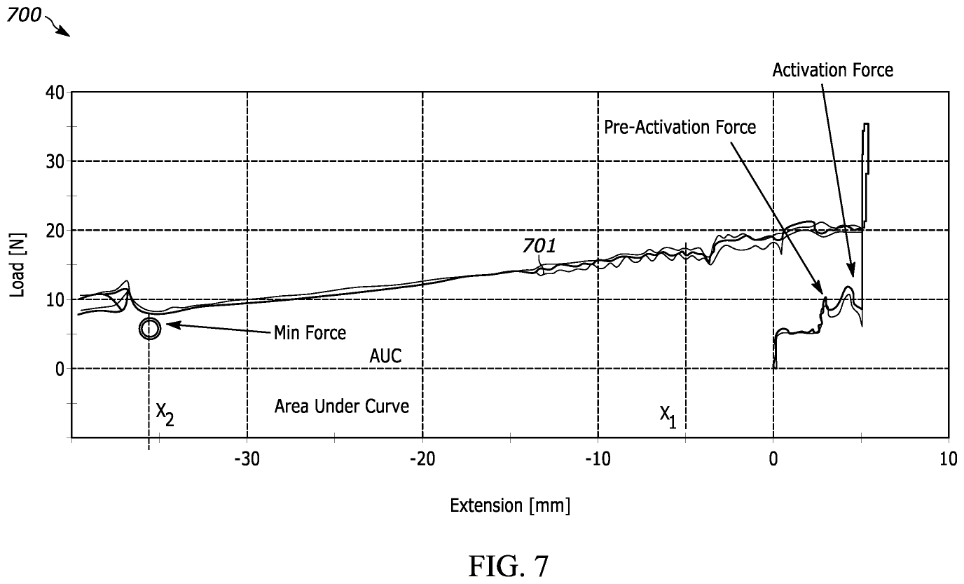

Turning to FIG. 7, an injection time (IT) model generation system 700 may include a RSA output force profile 701 measured using a syringe-free testing fixture 450 for 2.9 kgf RSA. Output force profile 701 of FIG. 7, collected using SyFR test fixture and parameterized using Eq. 2 may represent the force that a plunger rod of an RSA exerts on a plunger-stopper as a function of plunger rod displacement. The interval defined between x₁ and x₂ is the region of interest during DP extrusion and is used when calculating the IT. This interval is representative of the plunger rod extension starting at the point which plunger rod applies pressure on the plunger-stopper and ending at the point which plunger-stopper bottoms out on the cone area of the syringe (end of dose). The available energy the RSA exhibits for completing injection (injection energy) is defined as the AUC within the established interval. The AUC may be calculated by integrating the output force profile across this interval. The AUC is a factor contributing to the injection performance of the AI since it is strongly correlated with IT:

$${F}_{sp} ( x ) = {K}_{sp} ( {L}_{comp} - x ) \qquad Eq . 2$$

In Eq. 2, Fₛₚ(x) is the RSA force as a function of plunger rod displacement, Kₛₚ represents the RSA effective spring constant, and L_{comp} represents the RSA effective length of compression.

The minimum output force across the injection interval indicated in FIG. 7 may represent a minimum force that a plunger rod exerts on a plunger-stopper during drug product (DP) extrusion. A rear sub-assembly (RSA) may be, for example, configured to apply a minimum force to ensure that an associated autoinjector (AI) does not experience a stall during full dose injection.

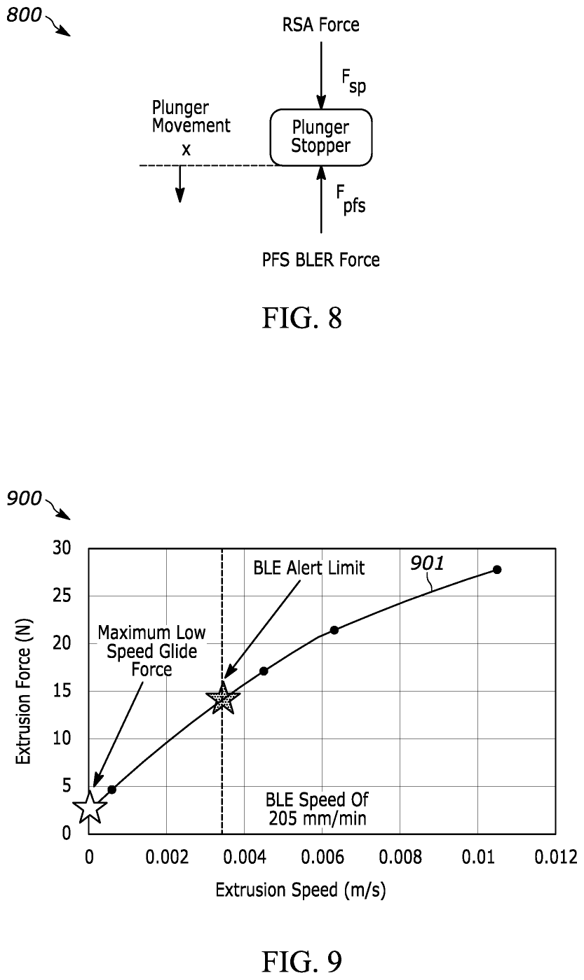

Mathematical modeling of injection time (IT) may operate by numerically solving a non-linear ordinary differential equation (Eq. 3) derived through a quasi-steady force balance analysis on the plunger-stopper of the PFS as described in FIG. 8. Eq. 3 may be derived by equating Eqs. 1 and 2. Note that the right-hand side of Equation comprises of constants that are introduced earlier in this document. The IT model provides the amount of time that it takes for the plunger-stopper to travel the required length in the syringe to extrude the full dose of DP from PFS.

F_{pfs} may be, for example, characterized using the BLER test method and parameterization of the PFS BLER profiles (e.g., profile 601 of FIG. 6, etc.)

Fₛₚ may be, for example, characterized using RSA output force test method and parameterization of the RSA output force profiles (e.g., profile 701 of FIG. 7, etc.):

$${{C}_{1} ( \frac{dx}{dt} )}^{2} + {C}_{2} \frac{dx}{dt} + {K}_{sp} x = {K}_{sp} {L}_{comp} - {F}_{f} \qquad Eq . 3$$

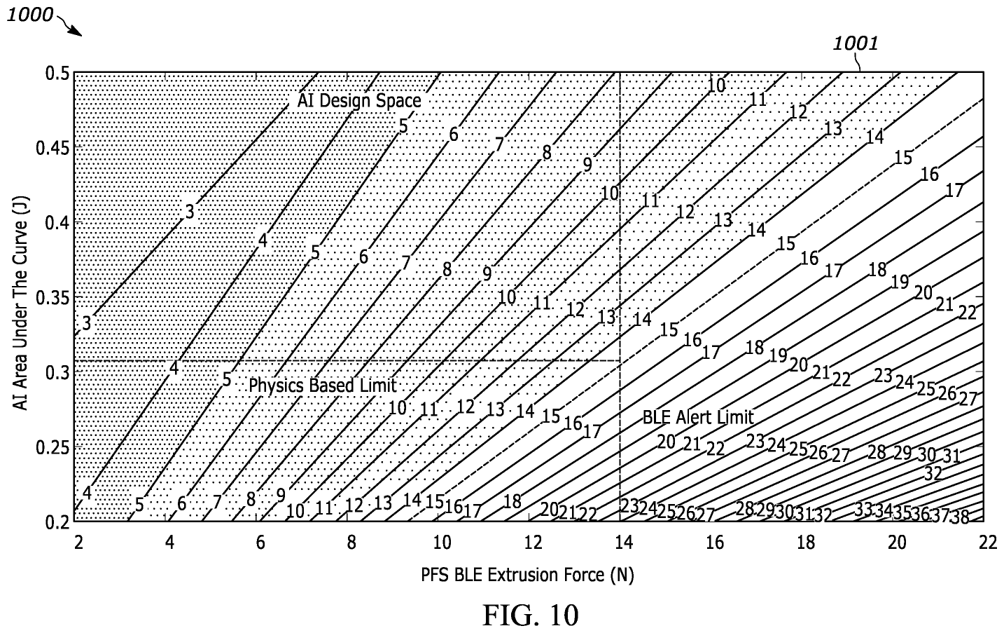

Turning to FIG. 8, an injection time (IT) model generation system 800 may implement a force balance analysis used for the IT model as illustrated by profile 801. Using the mathematical model of IT and 99% lower tolerance interval (TI) of the RSA performance (characterized using SyFR method, and parameterized using the maximum BLER profile that can extrude DP in IT of less than 15 seconds (if paired with 99% lower TI of RSA population (FIG. 7)) is characterized. From this hypothetical BLER profile, BLE (typically measured at extrusion speed of 205 mm/min per MET-406768) may be extracted and realized as the BLE alert limit of the DP PFS. Also, from this hypothetical maximum BLER profile, the maximum low speed glide force may be extracted. The graph 901 of FIG. 9 shows implementation of this methodology for a prefilled syringe (PFS) (e.g., a Repatha PFS, etc.) based on, for example, RSA data as provided in FIG. 10. As illustrated by the graph 1001, a BLE alert limit of 14 N and a maximum glide force of 2.5 N may be inferred, respectively.

With further reference to FIG. 9, a hypothetical maximum Repatha BLER profile 901 (scaled-up from FIG. 6) which will allow for an IT of less than 15 seconds if paired with 99% lower TI population of RSA profile (shown in FIG. 7) is illustrated. An extracted BLE alert limit is marked with an asterisk. An extracted maximum low speed glide force is also marked with an asterisk.

A minimum area under the curve (AUC) is defined to reach a desired injection time. Having the BLE alert limit established, a minimum energy (AUC) required for injection of DP with a specified IT for any PFS that shows BLE values less than the BLE alert limit then can be characterized using the IT model. Eq. 4 may provide a representation of IT as a function of AUC, C₁, C₂, F_{f}, and Δx (x₁−x₂).

With further reference to FIG. 10, characterization of the AUC required for extrusion of Repatha with IT of less than 15 seconds given that Repatha PFS exhibits BLE values smaller than the BLE alert limit of 14 N. Using the approach described herein, it is inferred that where BLE values less than the BLE alert limit are paired with RSAs that exhibit AUCs greater than the physics based limit derived using Eq. 4 (e.g., 0.31 J for a Repatha PFS, FIG. 10, etc.), then the injection time (IT) may be theoretically guaranteed to be less than an associated IT specification (e.g., 15 seconds for a Repatha PFS, FIG. 10, etc.). A design space may be identified for an autoinjector (AI) in order to robustly meet an associated IT specification given the PFS BLE and AUC:

$$t = \frac{2 Δ {xC}_{1}}{\sqrt{{C}_{2}^{2} + 4 {C}_{1} ( \frac{AUC}{Δ x} - {F}_{f} )} - {C}_{2}} \qquad Eq . 4$$

FIG. 10 depicts the minimum energy (AUC) for extrusion of Repatha with IT of less than 15 seconds is characterized as 0.31 J for all the PFSs that exhibit BLE values less than BLE alert limit of 14 N. An IT model for an AI may, for example, define minimum force to avoid injection stalls. The minimum force at the end of the injection interval (x₂) may be inferred as the least amount of force that the RSA can provide. Using the approach explained in section 2.d. the maximum low speed gliding force is characterized as the amount of force that is required to prevent plunger-stopper from coming to a stop during the injection. It follows that if the minimum force from FIG. 10 is greater than the maximum glide force from FIG. 9, avoidance of injection stalls may be guaranteed. Therefore, a maximum glide force from the hypothetical maximum BLER may be established as a minimum force needed by a RSA to avoid injection stalls.

The above description describes various devices, assemblies, components, subsystems and methods for use related to a drug delivery device such as a pre-filled syringe. The devices, assemblies, components, subsystems, methods or drug delivery devices (i.e., prefilled syringe) can further comprise or be used with a drug including but not limited to those drugs identified below as well as their generic and biosimilar counterparts. The term drug, as used herein, can be used interchangeably with other similar terms and can be used to refer to any type of medicament or therapeutic material including traditional and non-traditional pharmaceuticals, nutraceuticals, supplements, biologics, biologically active agents and compositions, large molecules, biosimilars, bioequivalents, therapeutic antibodies, polypeptides, proteins, small molecules and generics. Non-therapeutic injectable materials are also encompassed. The drug may be in liquid form, a lyophilized form, or in a reconstituted from lyophilized form. The following example list of drugs should not be considered as all-inclusive or limiting.

The drug will be contained in a reservoir within the pre-filled syringe for example. In some instances, the reservoir is a primary container that is either filled or pre-filled for treatment with the drug. The primary container can be a vial, a cartridge or a pre-filled syringe.

In some embodiments, the reservoir of the drug delivery device may be filled with or the device can be used with colony stimulating factors, such as granulocyte colony-stimulating factor (G-CSF). Such G-CSF agents include but are not limited to Neulasta® (pegfilgrastim, pegylated filgastrim, pegylated G-CSF, pegylated hu-Met-G-CSF) and Neupogen® (filgrastim, G-CSF, hu-MetG-CSF), UDENYCA® (pegfilgrastim-cbqv), Ziextenzo® (LA-EP2006; pegfilgrastim-bmez), or FULPHILA (pegfilgrastim-bmez).

In other embodiments, the drug delivery device may contain or be used with an erythropoiesis stimulating agent (ESA), which may be in liquid or lyophilized form. An ESA is any molecule that stimulates erythropoiesis. In some embodiments, an ESA is an erythropoiesis stimulating protein. As used herein, “erythropoiesis stimulating protein” means any protein that directly or indirectly causes activation of the erythropoietin receptor, for example, by binding to and causing dimerization of the receptor. Erythropoiesis stimulating proteins include erythropoietin and variants, analogs, or derivatives thereof that bind to and activate erythropoietin receptor; antibodies that bind to erythropoietin receptor and activate the receptor; or peptides that bind to and activate erythropoietin receptor. Erythropoiesis stimulating proteins include, but are not limited to, Epogen® (epoetin alfa), Aranesp® (darbepoetin alfa), Dynepo® (epoetin delta), Mircera® (methyoxy polyethylene glycol-epoetin beta), Hematide®, MRK-2578, INS-22, Retacrit® (epoetin zeta), Neorecormon® (epoetin beta), Silapo® (epoetin zeta), Binocrit® (epoetin alfa), epoetin alfa Hexal, Abseamed® (epoetin alfa), Ratioepo® (epoetin theta), Eporatio® (epoetin theta), Biopoin® (epoetin theta), epoetin alfa, epoetin beta, epoetin iota, epoetin omega, epoetin delta, epoetin zeta, epoetin theta, and epoetin delta, pegylated erythropoietin, carbamylated erythropoietin, as well as the molecules or variants or analogs thereof.

Among particular illustrative proteins are the specific proteins set forth below, including fusions, fragments, analogs, variants or derivatives thereof: OPGL specific antibodies, peptibodies, related proteins, and the like (also referred to as RANKL specific antibodies, peptibodies and the like), including fully humanized and human OPGL specific antibodies, particularly fully humanized monoclonal antibodies; Myostatin binding proteins, peptibodies, related proteins, and the like, including myostatin specific peptibodies; IL-4 receptor specific antibodies, peptibodies, related proteins, and the like, particularly those that inhibit activities mediated by binding of IL-4 and/or IL-13 to the receptor; Interleukin 1-receptor 1 (“IL1-R1”) specific antibodies, peptibodies, related proteins, and the like; Ang2 specific antibodies, peptibodies, related proteins, and the like; NGF specific antibodies, peptibodies, related proteins, and the like; CD22 specific antibodies, peptibodies, related proteins, and the like, particularly human CD22 specific antibodies, such as but not limited to humanized and fully human antibodies, including but not limited to humanized and fully human monoclonal antibodies, particularly including but not limited to human CD22 specific IgG antibodies, such as, a dimer of a human-mouse monoclonal hLL2 gamma-chain disulfide linked to a human-mouse monoclonal hLL2 kappa-chain, for example, the human CD22 specific fully humanized antibody in Epratuzumab, CAS registry number 501423-23-0; IGF-1 receptor specific antibodies, peptibodies, and related proteins, and the like including but not limited to anti-IGF-1R antibodies; B-7 related protein 1 specific antibodies, peptibodies, related proteins and the like (“B7RP-1” and also referring to B7H2, ICOSL, B7h, and CD275), including but not limited to B7RP-specific fully human monoclonal IgG2 antibodies, including but not limited to fully human IgG2 monoclonal antibody that binds an epitope in the first immunoglobulin-like domain of B7RP-1, including but not limited to those that inhibit the interaction of B7RP-1 with its natural receptor, ICOS, on activated T cells; IL-15 specific antibodies, peptibodies, related proteins, and the like, such as, in particular, humanized monoclonal antibodies, including but not limited to HuMax IL-15 antibodies and related proteins, such as, for instance, 145c7; IFN gamma specific antibodies, peptibodies, related proteins and the like, including but not limited to human IFN gamma specific antibodies, and including but not limited to fully human anti-IFN gamma antibodies; TALL-1 specific antibodies, peptibodies, related proteins, and the like, and other TALL specific binding proteins; Parathyroid hormone (“PTH”) specific antibodies, peptibodies, related proteins, and the like; Thrombopoietin receptor (“TPO-R”) specific antibodies, peptibodies, related proteins, and the like; Hepatocyte growth factor (“HGF”) specific antibodies, peptibodies, related proteins, and the like, including those that target the HGF/SF:cMet axis (HGF/SF:c-Met), such as fully human monoclonal antibodies that neutralize hepatocyte growth factor/scatter (HGF/SF); TRAIL-R2 specific antibodies, peptibodies, related proteins and the like; Activin A specific antibodies, peptibodies, proteins, and the like; TGF-beta specific antibodies, peptibodies, related proteins, and the like; Amyloid-beta protein specific antibodies, peptibodies, related proteins, and the like; c-Kit specific antibodies, peptibodies, related proteins, and the like, including but not limited to proteins that bind c-Kit and/or other stem cell factor receptors; OX40L specific antibodies, peptibodies, related proteins, and the like, including but not limited to proteins that bind OX40L and/or other ligands of the OX40 receptor; Activase® (alteplase, tPA); Aranesp® (darbepoetin alfa) Erythropoietin [30-asparagine, 32-threonine, 87-valine, 88-asparagine, 90-threonine], Darbepoetin alfa, novel erythropoiesis stimulating protein (NESP); Epogen® (epoetin alfa, or erythropoietin); GLP-1, Avonex® (interferon beta-1a); Bexxar® (tositumomab, anti-CD22 monoclonal antibody); Betaseron® (interferon-beta); Campath® (alemtuzumab, anti-CD52 monoclonal antibody); Dynepo® (epoetin delta); Velcade® (bortezomib); MLN0002 (anti-α4β7 mAb); MLN1202 (anti-CCR2 chemokine receptor mAb); Enbrel® (etanercept, TNF-receptor/Fc fusion protein, TNF blocker); Eprex® (epoetin alfa); Erbitux® (cetuximab, anti-EGFR/HER1/c-ErbB-1); Genotropin® (somatropin, Human Growth Hormone); Herceptin® (trastuzumab, anti-HER2/neu (erbB2) receptor mAb); Kanjinti™ (trastuzumab-anns) anti-HER2 monoclonal antibody, biosimilar to Herceptin®, or another product containing trastuzumab for the treatment of breast or gastric cancers; Humatrope® (somatropin, Human Growth Hormone); Humira® (adalimumab); Vectibix® (panitumumab), Xgeva® (denosumab), Prolia® (denosumab), Immunoglobulin G2 Human Monoclonal Antibody to RANK Ligand, Enbrel® (etanercept, TNF-receptor/Fc fusion protein, TNF blocker), Nplate® (romiplostim), rilotumumab, ganitumab, conatumumab, brodalumab, insulin in solution; Infergen® (interferon alfacon-1); Natrecor® (nesiritide; recombinant human B-type natriuretic peptide (hBNP); Kineret® (anakinra); Leukine® (sargamostim, rhuGM-CSF); LymphoCide® (epratuzumab, anti-CD22 mAb); Benlysta™ (lymphostat B, belimumab, anti-BlyS mAb); Metalyse® (tenecteplase, t-PA analog); Mircera® (methoxy polyethylene glycol-epoetin beta); Mylotarg® (gemtuzumab ozogamicin); Raptiva® (efalizumab); Cimzia® (certolizumab pegol, CDP 870); Soliris™ (eculizumab); pexelizumab (anti-C5 complement); Numax® (MEDI-524); Lucentis® (ranibizumab); Panorex® (17-1A, edrecolomab); Trabio® (lerdelimumab); TheraCim hR3 (nimotuzumab); Omnitarg (pertuzumab, 2C4); Osidem® (IDM-1); OvaRex® (B43.13); Nuvion® (visilizumab); cantuzumab mertansine (huC242-DM1); NeoRecormon® (epoetin beta); Neumega® (oprelvekin, human interleukin-11); Orthoclone OKT3® (muromonab-CD3, anti-CD3 monoclonal antibody); Procrit® (epoetin alfa); Remicade® (infliximab, anti-TNFα monoclonal antibody); Reopro® (abciximab, anti-GP IIb/IIIa receptor monoclonal antibody); Actemra® (anti-IL6 Receptor mAb); Avastin® (bevacizumab), HuMax-CD4 (zanolimumab); Mvasi™ (bevacizumab-awwb); Rituxan® (rituximab, anti-CD20 mAb); Tarceva® (erlotinib); Roferon-A®-(interferon alfa-2a); Simulect® (basiliximab); Prexige® (lumiracoxib); Synagis® (palivizumab); 145c7-CHO (anti-IL15 antibody, see U.S. Pat. No. 7,153,507); Tysabri® (natalizumab, anti-α4integrin mAb); Valortim® (MDX-1303, anti-B. anthracis protective antigen mAb); ABthrax™; Xolair® (omalizumab); ETI211 (anti-MRSA mAb); IL-1 trap (the Fc portion of human IgG1 and the extracellular domains of both IL-1 receptor components (the Type I receptor and receptor accessory protein)); VEGF trap (Ig domains of VEGFR1 fused to IgG1 Fc); Zenapax® (daclizumab); Zenapax® (daclizumab, anti-IL-2Ra mAb); Zevalin® (ibritumomab tiuxetan); Zetia® (ezetimibe); Orencia® (atacicept, TACI-Ig); anti-CD80 monoclonal antibody (galiximab); anti-CD23 mAb (lumiliximab); BR2-Fc (huBR3/huFc fusion protein, soluble BAFF antagonist); CNTO 148 (golimumab, anti-TNFα mAb); HGS-ETR1 (mapatumumab; human anti-TRAIL Receptor-1 mAb); HuMax-CD20 (ocrelizumab, anti-CD20 human mAb); HuMax-EGFR (zalutumumab); M200 (volociximab, anti-α5β1 integrin mAb); MDX-010 (ipilimumab, anti-CTLA-4 mAb and VEGFR-1 (IMC-18F1); anti-BR3 mAb; anti-C. difficile Toxin A and Toxin B C mAbs MDX-066 (CDA-1) and MDX-1388); anti-CD22 dsFv-PE38 conjugates (CAT-3888 and CAT-8015); anti-CD25 mAb (HuMax-TAC); anti-CD3 mAb (NI-0401); adecatumumab; anti-CD30 mAb (MDX-060); MDX-1333 (anti-IFNAR); anti-CD38 mAb (HuMax CD38); anti-CD40L mAb; anti-Cripto mAb; anti-CTGF Idiopathic Pulmonary Fibrosis Phase I Fibrogen (FG-3019); anti-CTLA4 mAb; anti-eotaxin1 mAb (CAT-213); anti-FGF8 mAb; anti-ganglioside GD2 mAb; anti-ganglioside GM2 mAb; anti-GDF-8 human mAb (MYO-029); anti-GM-CSF Receptor mAb (CAM-3001); anti-HepC mAb (HuMax HepC); anti-IFNα mAb (MEDI-545, MDX-198); anti-IGF1R mAb; anti-IGF-1R mAb (HuMax-Inflam); anti-IL12 mAb (ABT-874); anti-IL12/IL23 mAb (CNTO 1275); anti-IL13 mAb (CAT-354); anti-IL2Ra mAb (HuMax-TAC); anti-IL5 Receptor mAb; anti-integrin receptors mAb (MDX-018, CNTO 95); anti-IP10 Ulcerative Colitis mAb (MDX-1100); BMS-66513; anti-Mannose Receptor/hCGp mAb (MDX-1307); anti-mesothelin dsFv-PE38 conjugate (CAT-5001); anti-PD1mAb (MDX-1106 (ONO-4538)); anti-PDGFRa antibody (IMC-3G3); anti-TGFβ mAb (GC-1008); anti-TRAIL Receptor-2 human mAb (HGS-ETR2); anti-TWEAK mAb; anti-VEGFR/Flt-1 mAb; and anti-ZP3 mAb (HuMax-ZP3).

In some embodiments, the drug delivery device may contain or be used with a sclerostin antibody, such as but not limited to romosozumab, blosozumab, BPS 804 (Novartis), Evenity™ (romosozumab-aqqg), another product containing romosozumab for treatment of postmenopausal osteoporosis and/or fracture healing and in other embodiments, a monoclonal antibody (IgG) that binds human Proprotein Convertase Subtilisin/Kexin Type 9 (PCSK9). Such PCSK9 specific antibodies include, but are not limited to, Repatha® (evolocumab) and Praluent® (alirocumab). In other embodiments, the drug delivery device may contain or be used with rilotumumab, bixalomer, trebananib, ganitumab, conatumumab, motesanib diphosphate, brodalumab, vidupiprant or panitumumab. In some embodiments, the reservoir of the drug delivery device may be filled with or the device can be used with IMLYGIC® (talimogene laherparepvec) or another oncolytic HSV for the treatment of melanoma or other cancers including but are not limited to OncoVEXGALV/CD; OrienX010; G207, 1716; NV1020; NV12023; NV1034; and NV1042. In some embodiments, the drug delivery device may contain or be used with endogenous tissue inhibitors of metalloproteinases (TIMPs) such as but not limited to TIMP-3. In some embodiments, the drug delivery device may contain or be used with Aimovig® (erenumab-aooe), anti-human CGRP-R (calcitonin gene-related peptide type 1 receptor) or another product containing erenumab for the treatment of migraine headaches. Antagonistic antibodies for human calcitonin gene-related peptide (CGRP) receptor such as but not limited to erenumab and bispecific antibody molecules that target the CGRP receptor and other headache targets may also be delivered with a drug delivery device of the present disclosure. Additionally, bispecific T cell engager (BiTE®) antibodies such as but not limited to BLINCYTO® (blinatumomab) can be used in or with the drug delivery device of the present disclosure. In some embodiments, the drug delivery device may contain or be used with an APJ large molecule agonist such as but not limited to apelin or analogues thereof. In some embodiments, a therapeutically effective amount of an anti-thymic stromal lymphopoietin (TSLP) or TSLP receptor antibody is used in or with the drug delivery device of the present disclosure. In some embodiments, the drug delivery device may contain or be used with Avsola™ (infliximab-axxq), anti-TNF a monoclonal antibody, biosimilar to Remicade® (infliximab) (Janssen Biotech, Inc.) or another product containing infliximab for the treatment of autoimmune diseases. In some embodiments, the drug delivery device may contain or be used with Kyprolis® (carfilzomib), (2S)—N—((S)-1-((S)-4-methyl-1-((R)-2-methyloxiran-2-yl)-1-oxopentan-2-ylcarbamoyl)-2-phenylethyl)-2-((S)-2-(2-morpholinoacetamido)-4-phenylbutanamido)-4-methylpentanamide, or another product containing carfilzomib for the treatment of multiple myeloma. In some embodiments, the drug delivery device may contain or be used with Otezla® (apremilast), N-[2-[(1S)-1-(3-ethoxy-4-methoxyphenyl)-2-(methylsulfonyl)ethyl]-2,3-dihydro-1,3-dioxo-1H-isoindol-4-yl]acetamide, or another product containing apremilast for the treatment of various inflammatory diseases. In some embodiments, the drug delivery device may contain or be used with Parsabiv™ (etelcalcetide HCl, KAI-4169) or another product containing etelcalcetide HCl for the treatment of secondary hyperparathyroidism (sHPT) such as in patients with chronic kidney disease (KD) on hemodialysis. In some embodiments, the drug delivery device may contain or be used with ABP 798 (rituximab), a biosimilar candidate to Rituxan®/MabThera™, or another product containing an anti-CD20 monoclonal antibody. In some embodiments, the drug delivery device may contain or be used with a VEGF antagonist such as a non-antibody VEGF antagonist and/or a VEGF-Trap such as aflibercept (Ig domain 2 from VEGFR1 and Ig domain 3 from VEGFR2, fused to Fc domain of IgG1). In some embodiments, the drug delivery device may contain or be used with ABP 959 (eculizumab), a biosimilar candidate to Soliris®, or another product containing a monoclonal antibody that specifically binds to the complement protein C5. In some embodiments, the drug delivery device may contain or be used with Rozibafusp alfa (formerly AMG 570) is a novel bispecific antibody-peptide conjugate that simultaneously blocks ICOSL and BAFF activity. In some embodiments, the drug delivery device may contain or be used with Omecamtiv mecarbil, a small molecule selective cardiac myosin activator, or myotrope, which directly targets the contractile mechanisms of the heart, or another product containing a small molecule selective cardiac myosin activator. In some embodiments, the drug delivery device may contain or be used with Sotorasib (formerly known as AMG 510), a KRAS^{G12C} small molecule inhibitor, or another product containing a KRAS^{G12C} small molecule inhibitor. In some embodiments, the drug delivery device may contain or be used with Tezepelumab, a human monoclonal antibody that inhibits the action of thymic stromal lymphopoietin (TSLP), or another product containing a human monoclonal antibody that inhibits the action of TSLP. In some embodiments, the drug delivery device may contain or be used with AMG 714, a human monoclonal antibody that binds to Interleukin-15 (IL-15) or another product containing a human monoclonal antibody that binds to Interleukin-15 (IL-15). In some embodiments, the drug delivery device may contain or be used with AMG 890, a small interfering RNA (siRNA) that lowers lipoprotein(a), also known as Lp(a), or another product containing a small interfering RNA (siRNA) that lowers lipoprotein(a). In some embodiments, the drug delivery device may contain or be used with ABP 654 (human IgG1 kappa antibody), a biosimilar candidate to Stelara®, or another product that contains human IgG1 kappa antibody and/or binds to the p40 subunit of human cytokines interleukin (IL)-12 and IL-23. In some embodiments, the drug delivery device may contain or be used with Amjevita™ or Amgevita™ (formerly ABP 501) (mab anti-TNF human IgG1), a biosimilar candidate to Humira®, or another product that contains human mab anti-TNF human IgG1. In some embodiments, the drug delivery device may contain or be used with AMG 160, or another product that contains a half-life extended (HLE) anti-prostate-specific membrane antigen (PSMA) x anti-CD3 BiTE® (bispecific T cell engager) construct. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 119, or another product containing a delta-like ligand 3 (DLL3) CAR T (chimeric antigen receptor T cell) cellular therapy. In some embodiments, the drug delivery device may contain or be used with AMG 133, or another product containing a gastric inhibitory polypeptide receptor (GIPR) antagonist and GLP-1R agonist. In some embodiments, the drug delivery device may contain or be used with AMG 171 or another product containing a Growth Differential Factor 15 (GDF15) analog. In some embodiments, the drug delivery device may contain or be used with AMG 176 or another product containing a small molecule inhibitor of myeloid cell leukemia 1 (MCL-1). In some embodiments, the drug delivery device may contain or be used with AMG 199 or another product containing a half-life extended (HLE) bispecific T cell engager construct (BiTE®). In some embodiments, the drug delivery device may contain or be used with AMG 256 or another product containing an anti-PD-1 x IL21 mutein and/or an IL-21 receptor agonist designed to selectively turn on the Interleukin 21 (IL-21) pathway in programmed cell death-1 (PD-1) positive cells. In some embodiments, the drug delivery device may contain or be used with AMG 330 or another product containing an anti-CD33 x anti-CD3 BiTE® (bispecific T cell engager) construct. In some embodiments, the drug delivery device may contain or be used with AMG 404 or another product containing a human anti-programmed cell death-1 (PD-1) monoclonal antibody being investigated as a treatment for patients with solid tumors. In some embodiments, the drug delivery device may contain or be used with AMG 427 or another product containing a half-life extended (HLE) anti-fms-like tyrosine kinase 3 (FLT3) x anti-CD3 BiTE® (bispecific T cell engager) construct. In some embodiments, the drug delivery device may contain or be used with AMG 430 or another product containing an anti-Jagged-1 monoclonal antibody. In some embodiments, the drug delivery device may contain or be used with AMG 506 or another product containing a multi-specific FAP x 4-1BB-targeting DARPin® biologic under investigation as a treatment for solid tumors. In some embodiments, the drug delivery device may contain or be used with AMG 509 or another product containing a bivalent T-cell engager and is designed using XmAb® 2+1 technology. In some embodiments, the drug delivery device may contain or be used with AMG 562 or another product containing a half-life extended (HLE) CD19 x CD3 BiTE® (bispecific T cell engager) construct. In some embodiments, the drug delivery device may contain or be used with Efavaleukin alfa (formerly AMG 592) or another product containing an IL-2 mutein Fc fusion protein. In some embodiments, the drug delivery device may contain or be used with AMG 596 or another product containing a CD3 x epidermal growth factor receptor vIII (EGFRvIII) BiTE® (bispecific T cell engager) molecule. In some embodiments, the drug delivery device may contain or be used with AMG 673 or another product containing a half-life extended (HLE) anti-CD33 x anti-CD3 BiTE® (bispecific T cell engager) construct. In some embodiments, the drug delivery device may contain or be used with AMG 701 or another product containing a half-life extended (HLE) anti-B-cell maturation antigen (BCMA) x anti-CD3 BiTE® (bispecific T cell engager) construct. In some embodiments, the drug delivery device may contain or be used with AMG 757 or another product containing a half-life extended (HLE) anti-delta-like ligand 3 (DLL3) x anti-CD3 BiTE® (bispecific T cell engager) construct. In some embodiments, the drug delivery device may contain or be used with AMG 910 or another product containing a half-life extended (HLE) epithelial cell tight junction protein claudin 18.2 x CD3 BiTE® (bispecific T cell engager) construct.

Although the drug delivery devices, assemblies, components, subsystems and methods have been described in terms of exemplary embodiments, they are not limited thereto. The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein.

The detailed description is to be construed as exemplary only and does not describe every possible embodiment of the present disclosure. Numerous alternative embodiments could be implemented, using either current technology or technology developed after the filing date of this patent that would still fall within the scope of the claims defining the invention(s) disclosed herein. Those skilled in the art will recognize that a wide variety of modifications, alterations, and combinations can be made with respect to the above described embodiments without departing from the spirit and scope of the invention(s) disclosed herein, and that such modifications, alterations, and combinations are to be viewed as being within the ambit of the inventive concept(s).

1. A rear sub-assembly (RSA) output force characterization method, the method comprising:
providing a syringe-free (SyFR) rear sub-assembly (RSA) operating apparatus;
generating RSA output force profile data using the syringe-free (SyFR) rear sub-assembly (RSA) operating apparatus, wherein the RSA output force profile data is representative of a rear sub-assembly (RSA) output force profile; and
generating a RSA output energy profile based on the RSA output force profile data.

2. The method of claim 1, wherein the RSA output force profile data as a function of plunger rod displacement x is represented by:
$${F}_{sp} ( x ) = {K}_{sp} ( {L}_{comp} - x )$$
wherein Ksp represents a RSA effective spring constant, and wherein Lcomp represents a RSA effective length of compression.

3. The method of claim 1, wherein the rear sub-assembly (RSA) output force profile data is representative of an area under a curve (AUC).

4. The method of claim 1, wherein the rear sub-assembly (RSA) output force profile data is representative of a minimum force.

5. The method of claim 1, wherein the rear sub-assembly (RSA) output force profile data is representative of a pre-activation force.

6. The method of claim 1, wherein the rear sub-assembly (RSA) output force profile data is representative of an activation force.

7. An apparatus for characterizing output force of a drug delivery device or a rear subassembly of a drug delivery device, the apparatus comprising:
a driver holder configured to secure the drug delivery device or the rear subassembly to the apparatus, wherein the drug delivery device or the rear subassembly includes a drive member having a compressed position and an extended position;
a force sensor configured to measure output force data associated with the drive member as the drive member moves from the compressed position to the extended position;
an extrusion speed input configured to receive extrusion speed data, wherein the extrusion speed data is representative of a speed of the drive member; and
a controller configured to receive the output force data and the extrusion speed input data, and to generate an output force profile based on the output force data, wherein the controller is further configured to generate an output energy profile based on the output force profile data.

8. The rear sub-assembly (RSA) characterization apparatus as in claim 7, configured as a syringe-free (SyFR) rear sub-assembly fixture.

9. The rear sub-assembly (RSA) characterization apparatus as in claim 7, wherein the apparatus is configured to characterize a rear sub-assembly (RSA), wherein a RSA output force profile is proportional to a mechanical load, and wherein the controller is further configured to generate RSA characterization data based on a series of mechanical loads sequentially applied to the rear sub-assembly through a range of RSA plunger motion.

10. The rear sub-assembly (RSA) characterization apparatus as in claim 7, further comprising a rear sub-assembly (RSA) base.

11. The rear sub-assembly (RSA) characterization apparatus as in claim 7, further comprising a discoid configured to impart an inertial force on the RSA.

12. A non-transitory computer-readable medium storing computer-readable instructions that, when executed by a processor, causes the processor to generate an injection time (IT) model for an autoinjector, wherein execution of the instructions cause the processor to:
receive prefilled syringe (PFS) break loose and extrusion resistance (BLER) data, wherein the BLER data is representative of an amount of force required to achieve respective extrusion speeds;
receive rear sub-assembly (RSA) output force profile data; and
generate an injection time (IT) model for the autoinjector based on the BLER data and the RSA output force profile data.

13. The non-transitory computer-readable medium of claim 12, wherein the BLER data is representative of an amount of force required for extrusion of a drug product (DP) throughout a full dose injection

14. The non-transitory computer-readable medium of claim 12, wherein further execution of the instructions causes the processor to:
parameterize the prefilled syringe (PFS) break loose and extrusion resistance (BLER) data based on:
$${F}_{pfs} = {{C}_{1} ( \frac{dx}{dt} )}^{2} + {C}_{2} \frac{dx}{dt} + {F}_{f}$$
wherein
$$\frac{dx}{dt}$$
is representative of an extrusion speed, wherein C₁ represents a second order damping coefficient of the PFS, wherein C₂ represents a 1st order damping coefficient of the PFS, wherein Ff represents a low speed glide force of the PFS, and wherein C₂ is demonstrative of a non-Newtonian behavior of a drug product (DP) in the PFS.

15. The non-transitory computer-readable medium of claim 14, wherein, if C₂ is greater than zero, a shear-thickening behavior for the DP is inferred.

16. The non-transitory computer-readable medium of claim 14, wherein a shear-thinning behavior of the DP is inferred if C₂ is smaller than zero.

17. The non-transitory computer-readable medium of claim 14, if C₂ is approximately equal to zero, a Newtonian behavior for the DP is inferred.

18. The non-transitory computer-readable medium of claim 14, wherein F represents a low speed glide force of the PFS.

19. The non-transitory computer-readable medium of claim 14, wherein F is as an amount of force to keep a plunger-stopper traveling during injection.

20. The non-transitory computer-readable medium of claim 14, wherein F is an amount of force to avoid stall of AI.
